(19) United States
(12) Patent Application Publication
(10) Pub. No.: US 2025/0213602 A1
(43) Pub. Date: Jul. 3, 2025

(54) PHARMACEUTICAL COMPOSITIONS FOR DEMODEX RELATED BLEPHARITIS AND EYELID CRUSTING

(71) Applicant: Tarsus Pharmaceuticals, Inc., Irvine, CA (US)

(72) Inventors: Tiang Hin Jerry Tan, Singapore (SG); Yong Ming Por, Singapore (SG)

(21) Appl. No.: 19/087,897

(22) Filed: Mar. 24, 2025

Related U.S. Application Data
(63) Continuation of application No. 17/570,745, filed on Jan. 7, 2022, now Pat. No. 12,257,263.
Application No. 17/570,745 is a continuation of application No. 16/725,761, filed on Dec. 23, 2019, now abandoned.
Application No. 16/725,761 is a continuation of application No. 15/501,420, filed on Feb. 2, 2017, now abandoned.

(51) Int. Cl. A61K 31/7048; A61K 9/00; A61K 9/06; A61K 9/107; A61K 31/65; A61K 45/06; A61K 47/06; A61K 47/10; A61K 47/14; A61K 47/18; A61K 47/22; A61K 47/24; A61K 47/26; A61K 47/34; A61K 47/44

(52) U.S. Cl. CPC A61K 31/7048; A61K 9/0014; A61K 9/0048; A61K 9/06; A61K 9/107; A61K 31/65; A61K 45/06; A61K 47/06; A61K 47/10; A61K 47/14; A61K 47/183; A61K 47/22; A61K 47/24; A61K 47/26; A61K 47/34; A61K 47/44

(57) ABSTRACT

Formulation of ectoparasiticidal and antibiotic “compositions into pharmaceutical compositions useful for the treatment of eyelid inflammation, in particular demodex related blepharitis and eye crusting.

### TECHNICAL FIELD

The present invention relates to the formulation of ectoparasiticidal and antibiotic compositions into topical pharmaceutical compositions useful for the treatment of eyelid inflammation, in particular demodex related blepharitis and eyelid crusting. This invention also relates to a topical pharmaceutical composition suitable for the treatment of mammalian ectoparasites.

### DESCRIPTION OF BACKGROUND AND/OR RELATED AND/OR PRIOR ART

Blepharitis represents one of the most common anterior segment disorders encountered in ophthalmology. Blepharitis produces a red-rimmed appearance at the margins of the eyelids. It affects both the upper and lower eyelids. Blepharitis tends to recur and can become chronic.

Blepharitis is inflammation usually involving the part of the eyelid where the eyelashes grow. The eyes can eventually become red, itchy and irritated, with dandruff-like crusts appearing on the eyelashes (also known as “crusting”). It may also result in loss of lashes.

Recent studies show that ophthalmologists and optometrists observe blepharitis in approximately 37% to 47% of their patients. Despite the prevalence of blepharitis in both presentation and contribution to ocular conditions, there is little data on how eye care practitioners treat the disease.

A number of causes for blepharitis exist, including bacterial infections, parasitic infestation, viral infection and autoimmune conditions. Infective causes including bacteria and parasites are likely to be the most common causes. There are two types of blepharitis, i.e. anterior and posterior blepharitis. Anterior blepharitis is a condition where the outside front edge of the eyelids is inflamed, where the eyelashes are attached. Posterior blepharitis is a condition where the moist inside part of the eyelid is affected.

The traditional treatment for blepharitis consists of lid hygiene, and the application of a variety of “proprietary” cleansers. The goal is to control the disease and its underlying causes, maintain vision and to avoid secondary complications. However, many chronic cases persist. Demodex mites have been thought to be an etiologic factor in many cases of anterior blepharitis with lid margin debris. Demodex infestations are commonly treated with systemic and topical administration of parasiticides. For example, ocular demodex can be treated by performing a daily eyelid margin scrub with diluted shampoo alone or in combinations with a mercury oxide ointment, a metronidazole gel, or a pilocarpine gel applied to the base of the eye lashes. However, these treatments frequently fail to eradicate the demodex parasite and the infestation persists.

U.S. Pat. No. 8,128,968 teaches a composition containing about 0.6% to about 20% of tea tree oil to treat ocular demodex infestations and related conditions. Tea tree oil treatments, however, suffer from several disadvantages. Tea tree oil can easily lead to eye irritation and cause stinging sensations. Even a small amount will induce tearing, if it reaches the ocular surface. Due to the reflex tearing, dilution occurs and efficacy is reduced. Owing to the possibility of severe irritation, some eye centres carry out clinic based treatments, where the tea tree oil is professionally applied by an eye doctor. Tea tree oil compositions for home applications are thinner, which has little or perhaps no effect on the demodex mite population.

U.S. Pat. No. 5,952,372 teaches the use of treating demodex with oral administration of ivermectin. More specifically, oral ivermectin in a regimen of 200 micrograms per kilogram body weight per dose for 2 or 3 consecutive doses are given to the patient, at least 3 and not more than 7 days apart. Oral tetracycline was also given to the patients through consistent intermediate stages. However, this is an expensive medication regimen and requires repeated dosing. In our experience, a single dose, as used in some centers, does not eliminate the demodex mites. This is likely due to the pharmacokinetic properties of the ivermectin being partitioned into the various body compartments, with low concentrations at the specific target site.

Alternatively, U.S. Pat. No. 5,952,372 teaches topical ivermetin compounded to a 2% concentration by weight in a cream, lotion, or gel carrier vehicle as a treatment for all clinical stages and signs of rosacea in affected persons. However, this is only in relation to using a single active agent, ivermectin, for the treatment of a general dermatological condition such as rosacea, rather than specifically targeting blepharitis.

Demodex mites have also been found to be associated with various bacteria. including Bacillus oleronius. It is therefore possible that other bacteria play a role in the pathogenic process. Demodex mites killed by tea tree oil or via oral administration of ivermectin usually disintegrate on the ocular surface and release a variety of associated organisms, which probably explains why, in our experience, a proportion of patients continue to suffer the usual symptoms of blepharitis, despite eradication of the demodex mites.

### DETAILED DESCRIPTION

The aim of this invention is to overcome blepharitis, by eliminating demodex mites and/or other associated bacteria.

The dernodex species are microscopic, obligate, elongated mites which belong to the family “Demodicidae” of the order Acari of the class Arachnida. Demodex folliculorum and demodex brevis are found parasitizing on the human body surface. Demodex folliculorum occupies the hair follicles and upper sebaceous glands, whilst demodex brevis exists principally in the depth of the sebaceous glands [source: “A meta-analysis of association between acne vulgaris and Demodex infestation” by Ya-e Zhao, Li Hu, Li-Ping Wu, Jun-xian Ma, J Zhejiang Univ Sci B. 2012 March; 13(3):192-202].

This invention contains a parasiticidal agent such as ivermectin, and an antibiotic such as oxytetracycline, or any other tetracycline or macrolide antibiotic. Other types of antibiotics are also suitable, such as fluoroquinolones and aminoglycosides. Tetracycline antibiotics comprise a class of anti-microbials with applications in human and veterinary medicine, and are among the most heavily used antibiotics in the world. The purpose of antibiotics is to kill harmful bacteria, where possible, or to at least reduce their proliferation with bacteriostatic compounds.

The pharmaceutical composition according to the invention are suited for treating blepharitis and may be in liquid, pasty form, and more particularly in the form of creams, ointments, milks, pomades, powders, impregnated pads, syndets, towelettes, solutions, gels, sprays, foams, suspensions, lotions, sticks, shampoos, or washing bases.

In a preferred embodiment of this invention, the pharmaceutical composition according to the invention is in the form of an emulsion of the cream. In this embodiment, the compositions according to the invention are in the form of an emulsion. Most simple emulsions are oil-in-water, which means that the oil droplets are suspended in a continuous water phase, whereas others are water-in-oil. Emulsions can be classified as either oil-in-water (O/W) or water-in-oil (W/O) emulsions, depending on whether oil or water is the dispersed phase. Milk, cream and sauces are some examples of oil-in-water emulsions. O/W emulsion is preferred in this embodiment, where oil molecules are dispersed in water. Water therefore evaporates more readily from O/W emulsion. There are also more complicated emulsions that are used to enhance the delivery and stability of certain active ingredients. The choice of emulsification system is therefore highly dependent on the choice of ingredients comprised in the cream. Cream, regardless if it is cold cream, emollient cream, day cream, night cream, medicated cream, etc, all have the same basic emulsion formulation. The effectiveness of the cream further depends on the emulsion type and pH, as well as the type of oils, fats, alcohols and esters used.

In the above preferred embodiment of this invention, sorbitol is used as a humectant, so that the cream will not “dry out” when exposed to the atmosphere for prolonged periods of time. It also assists in maintaining phase stability, such as preventing separation of the aqueous and non-aqueous components.

The other Ingredients used in this embodiment of the invention are determined by referring to the Cosmetic Ingredient Review (“CIR”), or any other toxicological reports, where available, so as to ensure that these chosen ingredients are suitable for topical application and have lower risk of causing ocular irritation. The CIR was established in 1976 by the industry trade association (then the Cosmetic, Toiletry, and Fragrance Association, now the Personal Care Products Council, located in the United States of America), with the support of the U.S. Food and Drug Administration and the Consumer Federation of America. Although funded by the Council, CIR and the review process are independent from the Council and the cosmetics industry.

In this embodiment, emulsifying wax is used. There are several varieties of emulsifying wax available. Some are synthetically produced and some are vegetable-derived. This embodiment uses vegetable-based emulsifying wax NF (which means that it conforms to the specifications of the National Formulary, or “NF”), which is from naturally occurring fats and esters, derived from cetostearyl alcohol. Emulsifying wax (NF) is also suitable for most skin types as allergic reactions and skin sensitivities have rarely been recorded, in relation to such waxes.

According to the Final Report on the Safety Assessment of Fossil and Synthetic Waxes, International Journal of Toxicology (May/June 1983, 3: 43-99), concentrations of the Emulsifying Wax NF in the range of 1%-10% are found in typical use in skin cream. The ocular irritation of Emulsifying Wax NF was also studied in rabbits (according to the Draize method). The “Draize Test” is an acute toxicity test devised in 1944 by the Food and Drug Administration toxicologists John H. Draize and Jacob M. Spines. Initially used for testing cosmetics, the procedure involves applying 0.5 mL or 0.5 g of a test substance to the eye or skin of a restrained, conscious animal, and then leaving it for a set amount of time before rinsing it out and recording its effects. The Draize Test has since become an endorsed method to evaluate the safety of materials meant for use in or around the eyes.

According to the Amended Safety Assessment of Alkyl Esters as Used in Cosmetics, Cosmetics Ingredient Review (Apr. 12, 2013), Ethylhexyl Stearate is safe for use and the undiluted test material was at most mildly irritating to rabbit skin. In a 6-day cumulative skin irritation study, an undiluted test material (which had a mean maximum irritation index (MMII) of 0.67) was poorly tolerated, whereas a 10% aqueous solution (which had a MMII of 0.33) was relatively well tolerated. In human testing, a formulation containing 7.6% of Ethylhexyl Stearate was not an irritant or sensitizer (56 subjects), and not phototoxic (10 subjects), and not a photosensitizer (27 subjects), although some slight reactions were reported in the photosensitisation study. The undiluted test material did not provoke any significant injury in the rabbit's eyes (max Primary Irritation Index (PII) 4.67/100 at 1h). The Report stated that the reproductive toxicity of 2-ethyl-1-hexanol was already addressed in a fetotoxicity study (performed on diethylhexyl adipate); it was suggested that the fetotoxicity reported for mice in that study was actually due to a zinc deficiency and that given the extent of 2-ethyl-1-hexanol absorption and the load that would be expected to enter the hepatic circulation, the potential for 2-ethyl-1-hexanol-induced reproductive toxicity was not thought to be an issue.

According to the report: Safety Assessment of Cyclomethicone, Cyclotetrasiloxane, Cyclopentasiloxane, Cyclohexasiloxane, and Cycloheptasiloxane, International Journal of Toxicology (30 (Supplement 3)), rats were exposed to up to 700 ppm (0.07%) of Cyclopentasiloxane via inhalational (environmental) exposure for 5 days per week, for 12 months and 24 months respectively, and no eye lesions were found.

In the report: Safely Assessment of Tocopherols and Tocotrienols as Used in Cosmetics, Cosmetics Ingredient Review (Apr. 4, 2014), it was reported that tocopheryl acetate was not irritating to rabbit eyes in one study, but it produced weak to moderate conjunctival irritation in another study (European Chemicals Agency. 3,4-dihydro-2,5,7,8-tetramethyl-2-(4,8,12-trimethyldecyl)-2H-benzopyran-6-yl acetate). Undiluted tocopheryl acetate was instilled into the conjunctival sac of 3 Vienna White rabbits, and the eyes were not rinsed. The eyes were scored at 1, 24, 28, and 72 h after instillation. Slight irritation was observed at 1-48 h, and the eyes were normal at 72 h. In a modified Draize Test, the same protocol was followed, and undiluted dl-α-tocopherol was instilled into the eyes of six rabbits; again, the eyes were not rinsed. Weak to moderate conjunctival irritation (i.e., redness) was observed, which subsided by day 7. No corneal changes were reported.

Although mild irritation is shown in the studies by the European Chemical Agency (while some others showed no irritation), this embodiment of the invention uses diluted tocopheryl acetate.

According to the Final Report on the Safety Assessment of EDTA (Ethylenediaminetetraacetic acid), calcium disodium EDTA, diammonium EDTA, dipotassium EDTA, disodium EDTA, TEA-EDTA, tetrasodium EDTA, tripotassium EDTA, trisodium EDTA, HEDTA, and trisodium HEDTA, International Journal of Toxicology, (21 (Supplement 2); 95-142, 2002), Disodium EDTA was classified as a non-irritant in a primary mucous membrane irritation test using rabbit eyes. EDTA of approximately 0.5% to 1% is not toxic when added to balanced salt solutions.

According to the Final Report on the Safety Assessment of Methylisothiazolinone and Methylchloroisothiazlinone, Journal of the American College of Toxicology (Volume 11, Number 1, 1992), Methylisothiazolinone and Methylchloroisothiazlinone (MI/MCI) were evaluated for ocular irritation in eight Draize or modified Draize tests using albino rabbits. MI/MCI-886 ranging in concentration from 1.1% to 14% active ingredient (a.i.) and MI/MCI-GC with a 1.5% a.i. concentration were corrosive when tested as supplied. However, aqueous dilutions of MI/MCI-886 with concentrations of 0.056% a.i. were non-irritating: 0.28% a.i. was slight to moderately irritating; 0.56% and 1.7% a.i. were moderately to severely irritating; and 2.8% and 5.6% a.i. were severely irritating (corrosive).

As this invention is to be used topically, good absorption is achieved by using Propylene Glycol as a solvent for the ivermectin. According to the Cosmetic Ingredients Report Panel Meeting on Jun. 28-29, 2010, the ocular irritation of Propylene Glycol was determined using groups of 6 males and female New Zealand white albino rabbits. Undiluted Propylene Glycol was found to be a slight eye irritant. In this embodiment, Propylene Glycol is therefore diluted by approximately 10 times, and is therefore non-irritating to the eye, as seen in the our experimental studies on the patients.

The selection of the ingredients depends very much on the final purpose and the desired consistency (for example, creamy, hard, soft, greasy, or dry) of the invention. Changing one ingredient may also require changes in many others, if the physical characteristics of the invention are to be maintained.

Based on the abovementioned reports and scientific researches, various ingredients were selected for this invention.

According to one embodiment of the invention, the compositions comprise the following range (by weight):
ivermectin (1 to 5%)
an antibiotic such as oxytetracycline, and/or other tetracycline, macrolide, fluoroquinolone, or aminoglycoside antibiotic. (1 to 5%)
water (45 to 65%)
emulsifying wax NF (Cetearyl Alcohol, Polysorbate 60, PEG-150 Stearate, and Steareth-20) (5% to 15%)
ethylhexyl stearate (5% to 15%)
cyclopentasiloxane (0.01% to 0.05%)
sorbitol (1 to 10%)
tocopheryl acetate (0.01% to 0.5%)
disodium EDTA (0.01% to 0.1%)
propylene glycol (3% to 10%)
methylchloroisothiazolinone and methylisothiazolinone (0.001% to 0.01%).

The pharmaceutical composition according to the preferred embodiment is in the emulsion of a cream. In another embodiment, the invention is in the form of an ointment. A cream is a preparation of a medication for topical use that contains a water base. Essentially, it is a preparation of oil in water. An ointment is a preparation of a medication for topical use that contains an oil base, which is essentially a preparation of water in oil.

In another embodiment, the pharmaceutical composition according to the invention which is in the form of an ointment, which comprise the following range (by weight):
ivermectin (1-5%)
tetracycline (1-5%)
sorbitan monooleate (span 80) (3-5%)
light mineral oil (10%-15%)
petrolatum (70%-80%)

Whether in the form of a cream or ointment, the pharmaceutical composition according to the invention is therefore formulated in a way that it does not cause eye irritation, even if a small amount reaches the ocular surface. This invention therefore allows patients to apply such medication themselves, without having to go to a clinic. Patients may be able to continuously repeat the dosage over a period of time, so as to ensure a lower risk of recurrence of blepharitis.

In this invention, the topical application of the cream or ointment allows the ivermectin and antibiotic to be applied directly to the affected area, thus overcoming the problem associated with oral application of ivermectin being partitioned into the various body compartments, consequently with low concentrations at the specific target site. The direct topical application of the cream or ointment onto the affected area also minimises the risk of medication-related side effects felt in other parts of the body.

The cream or ointment is applied to the eyelids, in particular to eyelashes and the base of the eyelashes.

The cream or ointment may also be used to treat other conditions. It was mentioned in “A meta-analysis of association between acne vulgaris and Demodex infestation”, published in 2012, that there is evidence (including one meta-analysis) which suggests a connection between acne vulgaris and demodex. There is also other evidence which suggests a relationship between acne rosacea and demodex. For example, an earlier publication, Severe Demodex folliculorum-associated Oculocutaneous Rosacea In A Girl Successfully Treated With Ivermectin (JAMA Dermatol 2014 January; 150(1):61-3) reported the case of a 12-year old girl who was successfully treated with oral ivermectin. Other reports include Correlation Between Serum Reactivity To Demodex-associated Bacillus oleronius Proteins, and Altered Sebum Levels and Demodex Populations in Erythematotelangiectatic Rosacea Patients (J Med Microbiol 2014 February; 63 (Pt 2): 258-72, Evaluation of Demodex Folliculorum As A Risk Factor for the Diagnosis of Rosacea in Skin Biopsies (Indian J Dermatol. 2013 March, 58(2):157, Potential Role of Demodex Mites and Bacteria in the Induction of Rosacea (J Med Microbiol 2012 November; 61 (Pt 11): 1504-10), and Correlation Between Ocular Demodex Infestation and Serum Immunoreactivity to Bacillus Proteins in Patients with Facial Rosacea (Ophthalmology 2010 May: 117(5): 870-977). Accordingly, this invention may be used to treat other medical conditions as well.

The cream or ointment is topically administered every night to the affected areas, for the treatment of blepharitis and/or acne, to obtain the appropriate and desired treatment outcome, by using a clean finger or cotton bud. For example, the invention is to be applied in a light and gentle rubbing action across the base of the eyelashes by first closing the eyes and applying to the upper eyelid eyelash bases/roots, and then looking upwards so that the invention can be applied to the lower eyelid eyelash bases/roots. The invention is left on the affected area overnight.

The invention is then removed with normal cleansing water in the morning, for a month, so as to avoid early recurrences from new generations of mites which may emerge from unaffected eggs.

### EXAMPLES

The following examples are merely illustrative, and not limitative of the remainder of the disclosure in any way whatsoever. These examples are illustrative of the effectiveness of the invention.

Several patients had itching in the eyes and crusting around the eyelids. The presence of demodex mites was then confirmed. The confirmation was done by gently pulling on the eyelashes, and then transferring the presenting demodex mites onto a piece of cellophane tape. The said cellophane tape was then affixed onto a microscope slide. These demodex mites were viewed at 100 times of magnification, and counted to determine the mite density per lash follicle.

In treating the affected patients, the eyelids were first cleaned with hot compresses and a clean towel to remove all crusts and debris. The cream or ointment was then applied directly onto the affected area, by using a clean finger or a cotton-tipped applicator. The cream or ointment covered the entire eyelashes and roots.

The cream or ointment was left on the affected area for the entire night, and then washed away in the morning. This process was repeated on a nightly basis, for a month. The patients were then re-assessed for the presence of demodex mites, and no demodex mites were found on the eyelash roots of any patients. Before the treatment, many patients experienced itching on the eyelid margins. These patients were free of any symptoms, at the 1-month follow up.

Some cases were treated solely with ivermectin. However, these studies also demonstrate the necessity of antibiotics, in some cases.

### Example 1

This 52 year old Chinese gentleman had a long history of facial skin rashes that occurred along the nasolabial fold (also commonly known as “smile lines” or “laugh lines”) as well as around the ears. He had previously been treated by dermatologists for a diagnosis of seborrhoeic dermatitis with various creams including topical steroids. He presented with increasing crusting around the eyelashes and intermittent eye irritation. On examination, erythematous macules and papules were noted along the glabellar area as well as the nasolabial folds bilaterally. Marked crusting and many demodex mites were found in the eyelash follicles. A sample retrieved from one eyelash revealed 5 demodex folliculorum in the single follicle. He was treated with Ivermectin (only comprising 1%) cream nightly to the eyelid margins (as prescribed), as well as the inflamed areas of his face. One month later the eyelid margin crusting had resolved, as had the facial rash. No demodex mites were found in the eyelash follicles after the treatment.

### Example 2

This 44 year old Caucasian gentleman had recurring redness of the eyes associated with itching and burning sensations for the past 6 years. On examination, eyelash crusting together with lid margin erythema and many demodex mites were seen. He was first treated with oral Ivermectin (Stromectol) 12 mg and was advised to do scrubs using Tea Tree Oil on the eyelids twice a day. Over the next 2 months, the demodex mites reduced in numbers but were not completely eliminated, despite 4 subsequent doses of oral Ivermectin and continuing Tea Tree Oil scrubs. Tea Tree Oil applications were also performed on the patient. Owing to the persistence of the mites, dosages of Ivermectin (1%) cream was prescribed instead. After nightly applications of this cream for one month (as prescribed), no demodex mites were seen in the eyelash follicles and his ocular inflammation had markedly improved.

### Example 3

This 28 year old Chinese gentleman had recurring redness on the left eyelids for the past 9 years and had been treated previously with a variety of topical antibiotics including Framycetin. On examination, he had asymmetric disease, with severe inflammation of the left eyelid margins and comeal punctate erosions. Some demodex mites were seen in the eyelash follicles. He was treated with a combination of Ivermectin (1%) cream as well as Tetracycline eye ointment to the eyelids. Oral Doxycycline 100 mg twice a day was prescribed. Demodex mites resident in the lash follicles were eradicated after 1 month of nightly Ivermectin cream applications, as prescribed. Although eyelid inflammation was reduced, it was not entirely eliminated and the topical and oral tetracycline treatment was continued for the next 2 months. 4 months after initial presentation, the eyelids were much improved and inflammation had subsided. He was followed up for 2 years, and during this time remained free of severe episodes of eye or eyelid inflammation. A mild recurrence of demodex infestation was noted at the second year mark, and this was treated successfully with a further course of Ivermectin (1%) cream.

What is claimed is:

1. A method for the treatment of eyelid inflammation, in particular demodex related blepharitis and eyelid crusting, comprising topically administering every night for a month onto the affected area of an individual in need of such treatment and removed with normal cleansing water in the morning, a topical pharmaceutical composition which comprises an effective amount of ivermectin and an antibiotic, said topical pharmaceutical composition being formulated as an emulsion of a cream, the topical pharmaceutical cream comprising:
water;
an humectant comprising sorbitol;
a mixture of emollient comprising cyclopentasiloxane, ethylhexyl stearate;
an emulsifier comprising emulsifying wax NF;
a mixture of preservatives comprising methylchloroisothiazolinone, disodium EDTA;
an antioxidant comprising tocopheryl acetate; and
a solvent comprising propylene glycol.

2. The method defined by claim 1, wherein said ivermectin is present in the range of 1 to 5% (by weight) of the topical pharmaceutical composition.

3. The method defined by claim 1, wherein said antibiotic comprises of oxytetracycline in the range of 1% to 5% (by weight) of the topical pharmaceutical composition. 4 The method defined by claim 1, wherein said water is present in the range of 45% to 65% (by weight) of the topical pharmaceutical composition.

5. The method defined by claim 1, wherein said sorbitol is present in the range of 1% to 10% (by weight) of the topical pharmaceutical composition.

6. The method defined by claim 1, wherein said cyclopentasiloxane is present in the range of 0.01% to 0.05% (by weight) of the topical pharmaceutical composition.

7. The method defined by claim 1, wherein said ethylhexyl stearate is present in the range of 5% to 15% (by weight) of the topical pharmaceutical composition.

8. The method defined by claim 1, wherein said emulsifying wax NF is present in the range of 5% to 15% (by weight) of the topical pharmaceutical composition.

9. The method defined by claim 1, wherein said methylchloroisothiazolinone is present in the range of 0.001% to 0.01% (by weight) of the topical pharmaceutical composition.

10. The method defined by claim 1, wherein said disodium EDTA is present in the range of 0.01% to 0.1% (by weight) of the topical pharmaceutical composition,

11. The method defined by claim 1, wherein said tocopheryl acetate is present in the range of 0.01% to 0.5% (by weight) of the topical pharmaceutical composition.

12. The method defined by claim 1, wherein said propylene glycol is present in the range of 3% to 10% (by weight) of the topical pharmaceutical composition.

13. The method defined by claim 1 and claim 3, wherein said antibiotic can also comprise tetracycline.

14. The method defined by claim 1 and claim 3, wherein said antibiotic can also comprise a macrolide antibiotic.

15. The method defined by claim 1 and claim 3, wherein said antibiotic can also comprise fluoroquinolones.

16. The method defined by claim 1 and claim 3, wherein said antibiotic can also comprise aminoglycosides.

17. A method for the treatment of eyelid inflammation, in particular demodex related blepharitis and eye crusting, comprising topically administering every night for a month onto the affected area of an individual in need of such treatment and removed with normal cleansing water in the morning, a topical pharmaceutical composition which comprises a thus effective amount of ivermectin and an antibiotic comprising either tetracycline, macrolide, fluoroquinolone, or aminoglycoside, said topical pharmaceutical composition being formulated as an ointment, the topical pharmaceutical ointment comprising:
a solvent comprising sorbitan monooleate (span 80);
a mixture of ointment base comprising light mineral oil, petrolatum.

18. The method defined by claim 17 wherein said ivermectin is present in the range of 1% to 5% (by weight) of the topical pharmaceutical composition.

19. The method defined by claim 17 wherein said tetracycline is present in the range of 1% to 5% (by weight) of the topical pharmaceutical composition.

20. The method defined by claim 17 wherein said sorbitan monooleate is present in the range of 3% to 5% (by weight) of the topical pharmaceutical composition.

21. The method defined by claim 17 wherein said light mineral oil is present in the range of 10% to 15% (by weight) of the topical pharmaceutical composition.

22. The method defined by claim 17 wherein said petrolatum is present in the range of 70% to 80% (by weight) of the topical pharmaceutical composition.

23. A topically applicable stable pharmaceutical emulsion of cream, comprising:
ivermectin;
an antibiotic;
water;
an humectant comprising sorbitol;
a mixture of emollient comprising cyclopentasiloxane, ethylhexyl stearate, an emulsifier comprising emulsifying wax NF;
a mixture of preservatives comprising methylchloroisothiazolinone, disodium EDTA;
an antioxidant comprising tocopheryl acetate; and
a solvent comprising propylene glycol.

24. The topically applicable stable pharmaceutical emulsion of cream as defined by claim 23, wherein said ivermectin is present in the range of 1% to 5% (by weight) of the topical pharmaceutical composition.

25. The topically applicable stable pharmaceutical emulsion of cream as defined by claim 23, wherein said antibiotic is present in the range of 1% to 5% (by weight) of the topical pharmaceutical composition.

26. The topically applicable stable pharmaceutical emulsion of cream as defined by claim 23, wherein said water is present in the range of 45% to 65% (by weight) of the topical pharmaceutical composition.

27. The topically applicable stable pharmaceutical emulsion of cream as defined by claim 23, wherein said sorbitol is present in the range of 1% to 10% (by weight) of the topical pharmaceutical composition.

28. The topically applicable stable pharmaceutical emulsion of cream as defined by claim 23, wherein said cyclopentasiloxane is present in the range of 0.01% to 0.05% (by weight) of the topical pharmaceutical composition.

29. The topically applicable stable pharmaceutical emulsion of cream as defined by claim 23, wherein said ethylhexyl stearate is present in the range of 5% to 15% (by weight) of the topical pharmaceutical composition.

30. The topically applicable stable pharmaceutical emulsion of cream as defined by claim 23, wherein said emulsifying wax NF is present in the range of 5% to 15% (by weight) of the topical pharmaceutical composition.

31. The topically applicable stable pharmaceutical emulsion of cream as defined by claim 23, wherein said methylchloroisothiazolinone is present in the range of 0.001% to 0.01% (by weight) of the topical pharmaceutical composition. 32 The topically applicable stable pharmaceutical emulsion of cream as defined by claim 23, wherein said disodium EDTA is present in the range of 0.01% to 0.1% (by weight) of the topical pharmaceutical composition.

33. The topically applicable stable pharmaceutical emulsion of cream as defined by claim 23, wherein said tocopheryl acetate is present in the range of 0.01% to 0.5% (by weight) of the topical pharmaceutical composition.

34. The topically applicable stable pharmaceutical emulsion of cream as defined by claim 23, wherein said propylene glycol is present in the range of 3% to 10% (by weight) of the topical pharmaceutical composition.

35. The topically applicable stable pharmaceutical emulsion of cream as defined by claim 23 and claim 25, wherein said antibiotic can also comprise tetracycline.

36. The topically applicable stable pharmaceutical emulsion of cream as defined by claim 23 and claim 25, wherein said antibiotic can also comprise macrolide antibiotic.

37. The topically applicable stable pharmaceutical emulsion of cream as defined by claim 23 and claim 25, wherein said antibiotic can also comprise fluoroquinolones.

38. The topically applicable stable pharmaceutical emulsion of cream as defined by claim 23 and claim 25, wherein said antibiotic can also comprise aminoglycosides.

39. A topically applicable stable pharmaceutical in the form of an ointment, comprising:
ivermectin;
an antibiotic;
a solvent comprising sorbitan monooleate (span 80); and
a mixture of ointment base comprising light mineral oil, petrolatum.

40. The topically applicable stable pharmaceutical in the form of an ointment as defined by claim 39 wherein said ivermectin is present in the range of 1% to 5% (by weight) of the topical pharmaceutical composition.

41. The topically applicable stable pharmaceutical in the form of an ointment as defined by claim 39 wherein said antibiotic is present in the range of 1% to 5% (by weight) of the topical pharmaceutical composition.

42. The topically applicable stable pharmaceutical in the form of an ointment as defined by claim 39 wherein said sorbitan monooleate is present in the range of 3% to 5% (by weight) of the topical pharmaceutical composition.

43. The topically applicable stable pharmaceutical in the form of an ointment as defined by claim 39 wherein said light mineral oil is present in the range of 10% to 15% (by weight) of the topical pharmaceutical composition.

44. The topically applicable stable pharmaceutical in the form of an ointment as defined by claim 39 wherein said petrolatum is present in the range of 70% to 80% (by weight) of the topical pharmaceutical composition.

45. The topically applicable stable pharmaceutical in the form of an ointment as defined by claim 39 and claim 41, wherein said antibiotic can also comprise tetracycline.

46. The topically applicable stable pharmaceutical in the form of an ointment as defined by claims 39 and 41, wherein said antibiotic can also comprise macrolide antibiotic.

47. The topically applicable stable pharmaceutical in the form of an ointment as defined by claim 39 and claim 41, wherein said antibiotic can also comprise fluoroquinolones.

48. The topically applicable stable pharmaceutical in the form of an ointment as defined by claim 39 and claim 41, wherein said antibiotic can also comprise aminoglycosides.
